(19) United States
(12) Patent Application Publication
(10) Pub. No.: US 2025/0154335 A1
(43) Pub. Date: May 15, 2025

(54) CROSSLINKABLE ESTER AND URETHANE RESINS

(71) Applicant: THE BOARD OF REGENTS OF THE UNIVERSITY OF ALABAMA, Tuscaloosa, AL (US)

(72) Inventors: Jason Edward Bara, Tuscaloosa, AL (US); Mousumi Rani Bepari, Tuscaloosa, AL (US)

(21) Appl. No.: 18/828,101

(22) Filed: Sep. 9, 2024

Related U.S. Application Data
(60) Provisional application No. 63/597,516, filed on Nov. 9, 2023.

(51) Int. Cl. C08J 11/24; C07C 69/96; C07C 271/20; C07C 329/06; C07D 207/452

(52) U.S. Cl. CPC C08J 11/24; C07C 69/96; C07C 271/20; C07C 329/06; C07D 207/452

(57) ABSTRACT

Disclosed herein are crosslinkable ester and urethane resins and methods of making the same. Also disclosed are methods of recycling plastic waste by converting them to crosslinkable ester and urethane resins.

### CROSS REFERENCE TO RELATED APPLICATIONS

This application claims the benefit of priority to U.S. Provisional Application No. 63/597,516, filed Nov. 9, 2023, which is incorporated by reference herein in its entirety.

### ACKNOWLEDGEMENT OF GOVERNMENT FUNDING

This invention was made with government support under Grant No. 2132133 awarded by the National Science Foundation. The government has certain rights in the invention.

### BACKGROUND

Since the advent of the mass production of plastics for consumer goods in the early/mid-20th Century, just ˜9% of all plastic ever produced has been recycled. While collection and recycling rates have increased over time, ˜79% of all plastics ever made have ended up in landfills and/or have been released into natural environments. Furthermore, recycling processes (e.g., melting) are detrimental to the physical and mechanical properties and the resultant materials are generally inferior to virgin plastics due to oxidation and reductions in molecular weight (MW). This is in stark contrast to other common recyclables such as aluminum, where ˜50% of beverage cans are recycled annually and ˜75% of all aluminum produced since 1888 is still in use today, with virtually no difference in properties between recycled and new aluminum.

Once in the environment, plastic wastes are prone to form smaller particles known as microplastics, release toxic plasticizers and additives, and affect plant and animal life at all levels. The “Great Pacific Garbage Patch” is a notorious example of the excesses of our mismanagement and lack of attention of plastic waste. Polyethylene (PE), polypropylene (PP), poly(vinyl chloride) (PVC), and polyethylene terephthalate (PET) make up the large majority of this waste, and are thus the primary plastics polluting the environment. There remains a need for methods to “upcycle” (rather than recycle) these wastes into value-added products.

### SUMMARY

Disclosed herein are crosslinkable ester and urethane resins and methods of making the same. In an aspect, provided is a compound of Formula I:

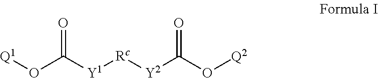

wherein each of Q¹ and Q² is independently selected at each occurrence from Formula IIa and Formula

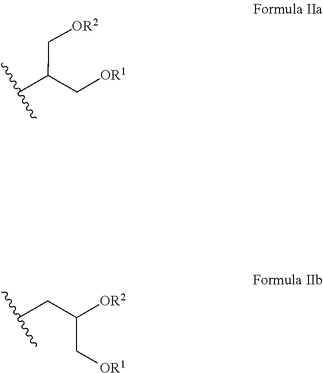

and wherein R^{c}, Y¹, Y², R¹, and R² are defined herein, and wherein, for at least one of Q¹ and Q², at least one of R¹ and R² can participate in a polymerization reaction. In some aspects each of Q¹ and Q² contains an R¹ and/or R² that can participate in a polymerization reaction

In another aspect, provided is a method of recycling a polymer into a resin/prepolymer comprising contacting a polymer with a compound of Formula IIIa or Formula IIIb:

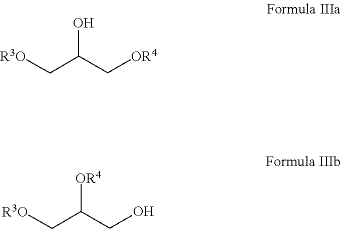

wherein R³ and R⁴ are defined herein, and wherein at least one of R³ and R⁴ can participate in a polymerization reaction.

In another aspect, provided is a method of recycling a polymer into a resin/prepolymer comprising a) contacting a polymer with a compound of Formula IV to give a first product:

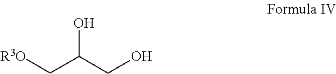

and b) contacting the first product with a compound of Formula V:

L—R⁴ Formula V

wherein R³, R⁴, and L are defined herein, and wherein R⁴ can participate in a polymerization reaction.

In another aspect, provided is a resin/prepolymer produced from any of the disclosed methods of recycling a polymer. In another aspect, provided is a method of producing a plastic comprising crosslinking the compound of Formula I.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### DETAILED DESCRIPTION

It is appreciated that certain features of the disclosure, which are, for clarity, described in the context of separate aspects, can also be provided in combination with a single aspect. Conversely, various features of the disclosure, which are, for brevity, described in the context of a single aspect, can also be provided separately or in any suitable subcombination. Unless defined otherwise, all technical and scientific terms used herein have the same meaning as commonly understood by one of ordinary skill in the art. Methods and materials similar or equivalent to those described herein can be used in the practice or testing of the present disclosure.

In this specification and in the claims that follow, reference will be made to a number of terms, which shall be defined to have the following meanings:

Throughout the description and claims of this specification, the word “comprise” and other forms of the word, such as “comprising” and “comprises,” means including but not limited to, and are not intended to exclude, for example, other additives, segments, integers, or steps. Furthermore, it is to be understood that the terms comprise, comprising, and comprises as they relate to various aspects, elements, and features of the disclosed invention also include the more limited aspects of “consisting essentially of” and “consisting of.”

As used herein, the singular forms “a,” “an,” and “the” include plural referents unless the context clearly dictates otherwise. Thus, for example, reference to a “polymer” includes aspects having two or more such polymers unless the context clearly indicates otherwise.

Ranges can be expressed herein as from “about” one particular value and/or to “about” another particular value. When such a range is expressed, another aspect includes from the one particular value and/or to the other particular value. Similarly, when values are expressed as approximations, by use of the antecedent “about,” it will be understood that the particular value forms another aspect. It should be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

For the terms “for example” and “such as,” and grammatical equivalences thereof, the phrase “and without limitation” is understood to follow unless explicitly stated otherwise.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

Compounds disclosed herein may be provided in the form of acceptable salts. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate.

The term “aliphatic” as used herein refers to an organic system composed of one or more alkyl, heteroalkyl, cycloalkyl, heterocycloalkyl, aryl, and heteroaryl groups in any permissible configuration. An aliphatic group may include any permissible number of substituents or degrees of unsaturation.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. By way of example, a C₁₋₆heteroalkyl) group includes, but is not limited to, the following structures:

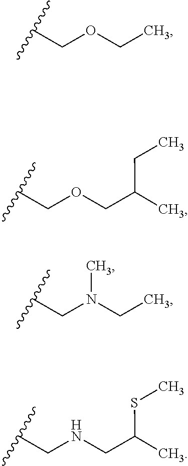

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halides, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkoxy” as used herein is an alkyl group bound through a single, terminal ether linkage; that is, an “alkoxy” group can be defined as—OW¹ where W¹ is alkyl as defined above.

The term “alkenyl” as used herein is a hydrocarbon group of from 2 to 24 carbon atoms with a structural formula containing at least one carbon-carbon double bond. Stereochemically undefined double bonds, unless specified to the contrary, are intended to include both the E and Z isomers. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl group can be substituted or unsubstituted. The aryl or heteroaryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

As used herein, the term “null,” when referring to a possible identity of a chemical moiety, indicates that the group is absent, and the two adjacent groups are directly bonded to one another. By way of example, for a genus of compounds having the formula CH₃—X—CH₃, if X is null, then the resulting compound has the formula CH₃—CH₃.

The term “halide,” “halo,” or “halogen” as used herein refers to the fluorine, chlorine, bromine, and iodine.

### Composition

In an aspect, provided is a compound of Formula I:

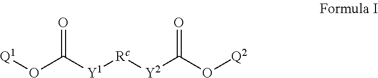

wherein: R^{c} is C₁₋₁₆aliphatic group; each of Y¹ and Y² is independently selected at each occurrence from null, NH, O, and S; each of Q¹ and Q² is independently selected at each occurrence from Formula IIa and Formula IIb:

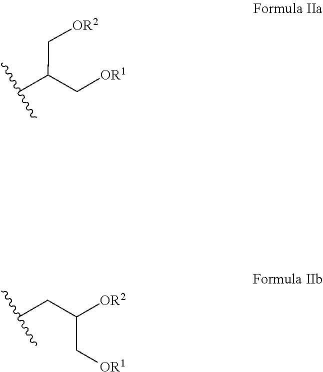

wherein R¹ is selected from R^{1a} or C(O)R^{1a}, wherein R^{1a} is selected from H, C₁₋₁₂ alkyl, C₂₋₁₂ alkenyl, C₂₋₁₂ alkynyl, carbocyclyl, aryl, heterocyclyl, or heteroaryl; wherein R^{1a} may be substituted one or more times by F, Cl, Br, I, OH, SH, C(═O)OₓR^{1b}, aryl, heteroaryl, N₃, or N(R^{1b})₂, wherein x is 0 or 1, R^{1b} is independently selected from H, C₁₋₄ alkyl, and C₂₋₄ alkenyl;

R² is selected from R^{2a} or C(O)R^{1a}, wherein R^{2a} is selected from H, C₁₋₁₂ alkyl, C₂₋₁₂ alkenyl, C₂-12 alkynyl, carbocyclyl, aryl, heterocyclyl, or heteroaryl; wherein R^{2a} may be substituted one or more times by F, Cl, Br, I, OH, SH, C(═O)O_{y}R^{2b}, aryl, heteroaryl, N₃, or N(R^{2b})₂, wherein y is 0 or 1, R^{2b} is independently selected from H, C₁₋₄ alkyl, and C₂₋₄ alkenyl; wherein, for at least one of Q¹ and Q², at least one of R¹ and R² can participate in a polymerization reaction.

In some aspects Y¹ and Y² are the same. In some aspects Y¹ and Y² are both O. In some aspects Y¹ and Y² are both NH. In some aspects Y¹ and Y² are both null.

In some aspects, R¹ has the formula:

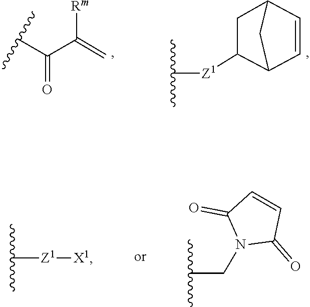

wherein R^{m} is selected from H and CH₃; Z¹ is selected from null, C₁₋₈ alkylene or C₁₋₈ heteroalkylene; X¹ is selected from CH═CH₂, C≡CH, N₃, SH; and R² is H or C₁₋₄ alkyl, optionally substituted one or more times by F, Cl, C₁₋₄ alkoxy, or aryl. In some aspects Z¹ is CH₂, CH₂CH₂, CH₂CH₂CH₂, CH₂CH₂O, or CH₂CH₂CH₂O.

In some aspects, R² has the formula:

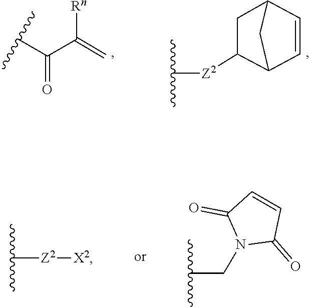

wherein R^{m} is selected from H and CH₃; Z² is selected from null, C₁₋₈ alkylene or C₁₋₈ heteroalkylene; X² is selected from CH═CH₂, C≡CH, N₃, SH; and R¹ is C₁₋₄ alkyl, optionally substituted one or more times by F, Cl, C₁₋₄ alkoxy, or aryl. In some aspects Z² is CH₂, CH₂CH₂, CH₂CH₂CH₂, CH₂CH₂O, or CH₂CH₂CH₂O.

In some aspects, R¹ has the formula:

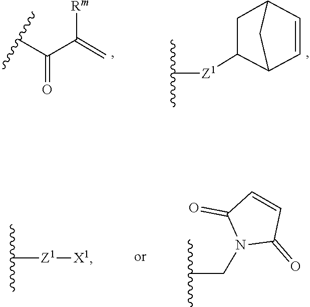

wherein R^{m} is selected from H and CH₃; Z¹ is selected from null, C₁₋₈ alkylene or C₁₋₈ heteroalkylene; X¹ is selected from CH═CH₂, C≡CH, N₃, SH; and R² has the formula:

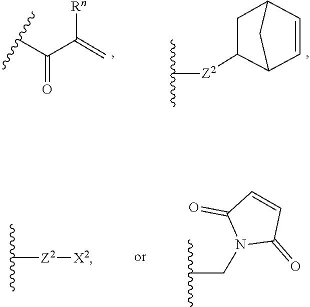

wherein Rⁿ is selected from H and CH₃; Z² is selected from null, C₁₋₈ n-alkylene; X² is selected from CH═CH₂, C≡CH, N₃, SH. In some aspects Z² is CH₂, CH₂CH₂, CH₂CH₂CH₂, CH₂CH₂O, or CH₂CH₂CH₂O.

In some aspects, R¹ is:

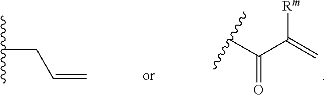

In some aspects, R² is:

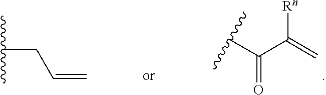

In some aspects, Y¹ and Y² are both null, or Y¹ and Y² are both NH.

In some aspects, R^{c} is C₂₋₈ n-alkyl or C₂₋₈ alkenyl.

In some aspects R^{c} has the formula:

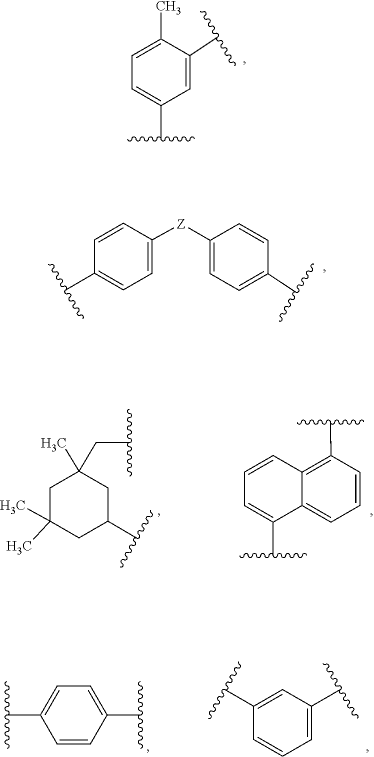

wherein Z is selected from null, CH₂, C(CH₃)₂, O, or NH.

In some aspects, R^{c} is

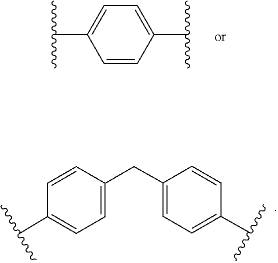 [SMILES: *c1ccc(*)cc1 ; *c1ccc(Cc2ccc(*)cc2)cc1]

In some aspects, the compound of Formula I is selected from:

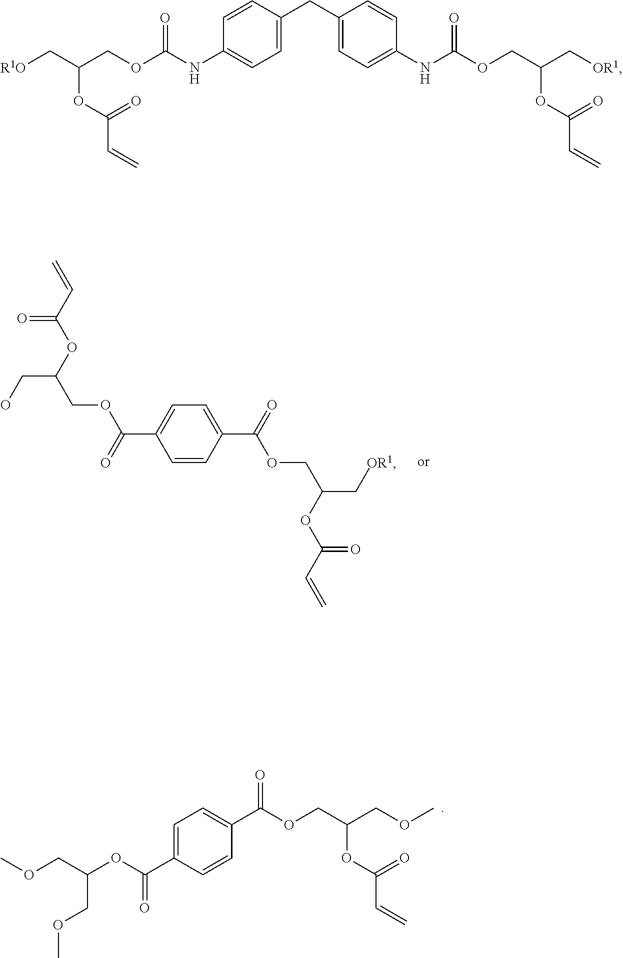

In certain aspects, R¹ is C₁₋₄ alkyl. In some aspects R¹ is methyl, trifluoromethyl, benzyl, 4-methoxybenzyl, or 2-methoxyethyl.

### Methods

In an aspect, provided is a method of recycling a polymer into a resin/prepolymer (e.g., a compound of Formula I) comprising contacting a polymer with a compound of Formula IIIa or Formula IIIb:

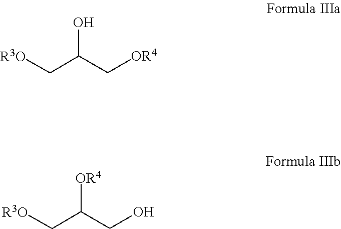

wherein R³ is selected from R^{3a} or C(O)R^{3a}, R⁴ is selected from R^{4a} or C(O)R^{4a}, R^{3a} is selected from H, C₁₋₁₂ alkyl, C₂₋₁₂ alkenyl, C₂₋₁₂ alkynyl, aryl, heterocyclyl, or heteroaryl; wherein R^{3a} may be substituted one or more times by F, Cl, Br, I, OH, SH, C(═O)OₓR^{3b}, aryl, heteroaryl, N₃, or N(R^{3b})₂, wherein x is 0 or 1, R^{3b} is independently selected from H, C₁₋₄ alkyl, and C₂₋₄ alkenyl; R^{4a} is selected from H, C₁₋₁₂ alkyl, C₂₋₁₂ alkenyl, C₂₋₁₂ alkynyl, aryl, heterocyclyl, or heteroaryl; wherein R^{4a} may be substituted one or more times by F, Cl, Br, I, OH, SH, C(═O)O_{y}R^{4b}, aryl, heteroaryl, N₃, or N(R^{4b})₂, wherein y is 0 or 1, R^{4b} is independently selected from H, C₁₋₄ alkyl, and C₂₋₄ alkenyl; and wherein at least one of R³ or R⁴ can participate in a polymerization reaction. In certain implementations, neither of R³ or R⁴ are hydrogen.

In another aspect, provided is a method of recycling a polymer into a resin/prepolymer (e.g., a compound of Formula I) comprising a) contacting a polymer with a compound of Formula IV:

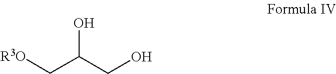

and b) contacting a product of the polymer and the compound of Formula IV with a compound of Formula V:

L—R⁴ Formula V

wherein R³ is selected from R^{3a} or C(O)R^{3a}, R⁴ is selected from R^{4a} or C(O)R^{4a}, L is a leaving group, R^{3a} is selected from H, C₁₋₁₂ alkyl, C₂₋₁₂ alkenyl, C₂₋₁₂ alkynyl, aryl, heterocyclyl, or heteroaryl; wherein R^{3a} may be substituted one or more times by F, Cl, Br, I, OH, SH, C(═O)OₓR^{3b}, aryl, heteroaryl, N₃, or N(R^{3b})₂, wherein x is 0 or 1, R^{3b} is independently selected from H, C₁₋₄ alkyl, and C₂₋₄ alkenyl; R^{4a} is selected from C₁₋₁₂ alkyl, C₂₋₁₂ alkenyl, C₂₋₁₂ alkynyl, aryl, heterocyclyl, or heteroaryl; wherein R^{4a} may be substituted one or more times by F, Cl, Br, I, OH, SH, C(═O)O_{y}R^{4b}, aryl, heteroaryl, N₃, or N(R^{4b})₂, wherein y is 0 or 1, R^{4b} is independently selected from H, C₁₋₄ alkyl, and C₂₋₄ alkenyl; and wherein R⁴ can participate in a polymerization reaction.

In some aspects, the method of recycling a polymer occurs via:

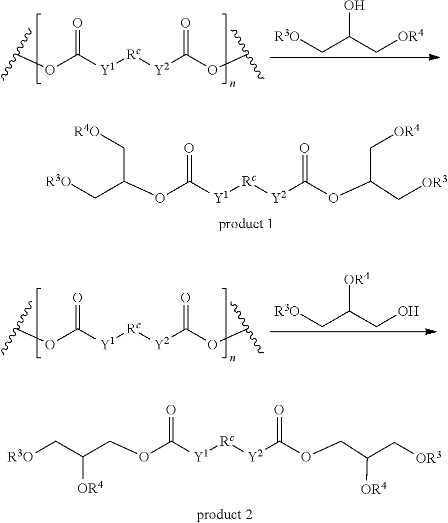

In some aspects, only one of the compound of Formula IIIa and the compound of Formula IIIb is used in the method to produce only product 1 or only product 2, respectively. In some aspects, both the compound of Formula IIIa and the compound of Formula IIIb are used in the method to product a mixture of product 1 and product 2. In some aspects, the product of the method is at least 40 mol % product 1, or at least 45 mol % product 1, or at least 50 mol % product 1, or at least 55 mol % product 1, or at least 60 mol % product 1, or at least 65 mol % product 1, or at least 70 mol % product 1, or at least 75 mol % product 1, or at least 80 mol % product 1, or at least 85 mol % product 1, or at least 90 mol % product 1, or at least 95 mol % product 1, relative to the total of products 1 and 2.

In some aspects, the method of recycling a polymer occurs via:

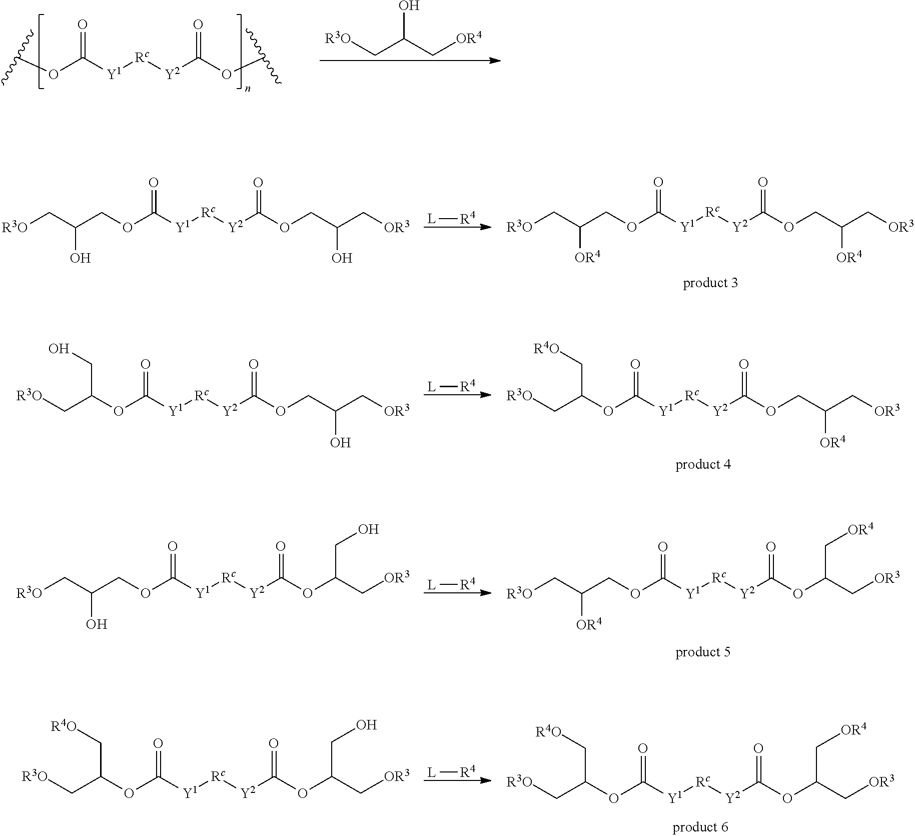

In some aspects, the product of the method of recycling a polymer is at least 40 mol % product 3, or at least 45 mol % product 3, or at least 50 mol % product 3, or at least 55 mol % product 3, or at least 60 mol % product 3, or at least 65 mol % product 3, or at least 70 mol % product 3, or at least 75 mol % product 3, or at least 80 mol % product 3, or at least 85 mol % product 3, or at least 90 mol % product 3, or at least 95 mol % product 3, relative to the total of products 3, 4, 5, and 6.

In some aspects, R³ has the formula:

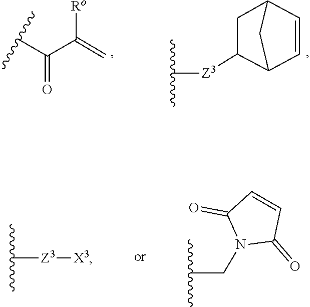

wherein R^{o} is selected from H and CH₃; Z³ is selected from null, C₁₋₈ alkylene or C₁₋₈ heteroalkylene; X³ is selected from CH═CH₂, C≡CH, N₃, SH; and R⁴ is H or C₁₋₄ alkyl, optionally substituted one or more times by F, Cl, C₁₋₄ alkoxy, or aryl. In some aspects Z³ is CH₂, CH₂CH₂, CH₂CH₂CH₂, CH₂CH₂O, or CH₂CH₂CH₂O.

In some aspects, R⁴ has the formula:

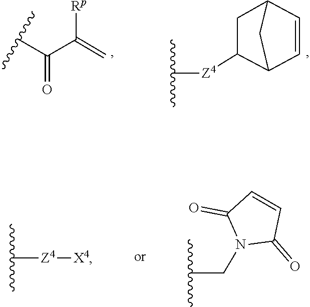

wherein R^{p} is selected from H and CH₃; Z⁴ is selected from null, C₁₋₈ alkylene or C₁₋₈ heteroalkylene; X⁴ is selected from CH═CH₂, C≡CH, N₃, SH; and R³ is C₁₋₄ alkyl, optionally substituted one or more times by F, Cl, C₁₋₄ alkoxy, or aryl. In some aspects Z⁴ is CH₂, CH₂CH₂, CH₂CH₂CH₂, CH₂CH₂O, or CH₂CH₂CH₂O.

In some aspects, R³ has the formula:

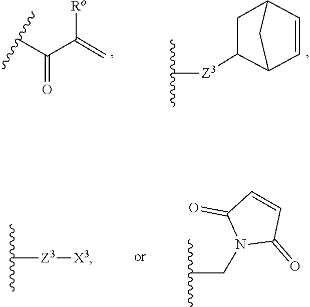

wherein R^{o} is selected from H and CH₃; Z³ is selected from null, C₁₋₈ alkylene or C₁₋₈ heteroalkylene; X³ is selected from CH═CH₂, C≡CH, N₃, SH; and R⁴ has the formula:

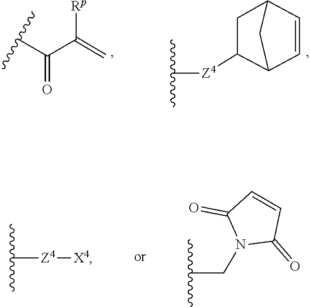

wherein R^{p} is selected from H and CH₃; Z⁴ is selected from null, C₁₋₈ alkylene or C₁₋₈ heteroalkylene; X⁴ is selected from CH═CH₂, C≡CH, N₃, SH.

In some aspects, R³ is

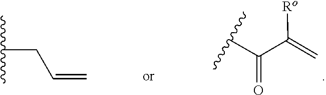

In some aspects, R⁴ is

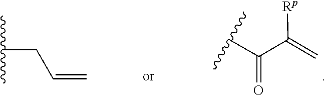

In some aspects, the compound of Formula V is selected from:

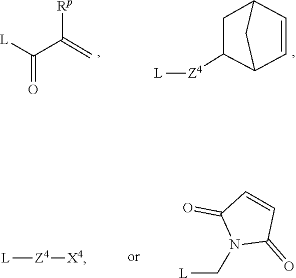

wherein L represents a leaving group, R^{p} is selected from H and CH₃; Z⁴ is selected from null, C₁₋₈ n-alkylene; X⁴ is selected from CH═CH₂, C≡CH, N₃, SH; and R³ is C₁₋₄ alkyl, optionally substituted one or more times by F, Cl, C₁₋₄ alkoxy, or aryl.

In some aspects, the compound of Formula V is

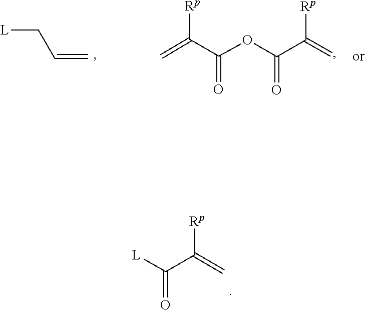

In some aspects, L is OH, CI, Br, I, OC₆F₅, OSOR^{s}, wherein R^{s} is methyl, trifluoromethyl, or 4-methylphenyl, or another suitable leaving group, which could be identified by one of ordinary skill in the art. In some aspects, Lis OH and a transesterification reaction occurs between one of the OH groups of the compound of Formula IV and the compound of Formula V.

In some aspects, the polymer comprises polyethylene terephthalate, polyester, polyurethane, polyesteramide, polyamide, epoxy resin, or any combination thereof.

In some aspects, the compound of Formula IIIa and/or IIIb is a liquid and further serves as a solvent for the recycling process. The compound can be a liquid at room temperature or at the temperature of the recycling process In certain aspects, the polymer is combined with liquid compound of Formula IIIa and/or IlIb in an amount that is from 1-50 wt. %, from 1-25 wt. %, from 1-10 wt. %, from 1-5 wt. %, from 5-10 wt. %, from 5-25 wt. %, from 10-25 wt. %, from 20-40 wt. %, or from 25-50 wt. %.

In some aspects, the recycling process can be conducted in the presence of an additional solvent. In some implementations, the solvent can be a C₆₋₁₂hydrocarbon carbon solvent, e.g., heptanes, xylenes, etc. In some implementations the solvent can be a polar, aprotic solvent, e.g., acetone, DMSO, DMF, DMA, acetonitrile, and the like.

In some aspects, the compound of Formula IIIa is present, relative to the polymer, in an amount of 100-1000 wt. %, or 150-950 wt. %, or 200-900 wt. %, or 250-850 wt. %, or 300-800 wt. %, or 350-750 wt. %, or 400-700 wt. %, or 450-650 wt. %, or 500-600 wt. %, or 100-500 wt. %, or 150-450 wt. %, or 200-400 wt. %, or 250-350 wt. %, or 500-1000 wt. %, or 550-950 wt. %, or 600-900 wt. %, or 650-85 wt.0%, or 700-800 wt. %, and the compound of Formula IIIb is absent. In some aspects, the compound of Formula IIIb is present in an amount, relative to the polymer, of 100-1000 wt. %, or 150-950 wt. %, or 200-900 wt. %, or 250-850 wt. %, or 300-800 wt. %, or 350-750 wt. %, or 400-700 wt. %, or 450-650 wt. %, or 500-600 wt. %, or 100-500 wt. %, or 150-450 wt. %, or 200-400 wt. %, or 250-350 wt. %, or 500-1000 wt. %, or 550-950 wt. %, or 600-900 wt. %, or 650-850 wt. %, or 700-800 wt. % relative to the polymer, and the compound of Formula IIIa is absent. In some aspects, the compound of Formula IIIa is present in an amount of 10-1000 wt. %, or 20-950 wt. %, or 40-900 wt. %, or 60-850 wt. %, or 80-800 wt. %, or 100-750 wt. %, or 150-700 wt. %, or 200-650 wt. %, or 250-600 wt. %, or 300-550 wt. %, or 350-500 wt. %, or 400-450 wt. %, or 10-400 wt. %, or 20-350 wt. %, or 40-300 wt. %, or 60-250 wt. %, or 80-200 wt. %, or 100-150 wt. %, or 450-1000 wt. %, or 500-950 wt. %, or 550-900 wt. %, or 600-850 wt. %, or 650-800 wt. %, or 700-750 wt. % relative to the polymer, and the compound of Formula IIIb is present in an amount of 10-1000 wt. %, or 20-950 wt. %, or 40-900 wt. %, or 60-850 wt. %, or 80-800 wt. %, or 100-750 wt. %, or 150-700 wt. %, or 200-650 wt. %, or 250-600 wt. %, or 300-550 wt. %, or 350-500 wt. %, or 400-450 wt. %, or 10-400 wt. %, or 20-350 wt. %, or 40-300 wt. %, or 60-250 wt. %, or 80-200 wt. %, or 100-150 wt. %, or 450-1000 wt. %, or 500-950 wt. %, or 550-900 wt. %, or 600-850 wt. %, or 650-800 wt. %, or 700-750 wt. % relative to the polymer.

In some aspects, the compound of Formula IV is present in an amount of 100-1000 wt. %, or 150-950 wt. %, or 200-900 wt. %, or 250-850 wt. %, or 300-800 wt. %, or 350-750 wt. %, or 400-700 wt. %, or 450-650 wt. %, or 500-600 wt. %, or 100-500 wt. %, or 150-450 wt. %, or 200-400 wt. %, or 250-350 wt. %, or 500-1000 wt. %, or 550-950 wt. %, or 600-900 wt. %, or 650-850 wt. %, or 700-800 wt. % relative to the polymer. In some aspects the compound of Formula V is present in an amount of 10-1000 wt. %, or 20-950 wt. %, or 40-900 wt. %, or 60-850 wt. %, or 80-800 wt. %, or 100-750 wt. %, or 150-700 wt. %, or 200-650 wt. %, or 250-600 wt. %, or 300-550 wt. %, or 350-500 wt. %, or 400-450 wt. %, or 10-400 wt. %, or 20-350 wt. %, or 40-300 wt. %, or 60-250 wt. %, or 80-200 wt. %, or 100-150 wt. %, or 450-1000 wt. %, or 500-950 wt. %, or 550-900 wt. %, or 600-850 wt. %, or 650-800 wt. %, or 700-750 wt. % relative to the polymer.

In some aspects, the method of recycling a polymer is performed at a temperature of from about 30° C. to about 200° C., or about 50° C. to about 180° C., or about 70° C. to about 160° C., or about 90° C. to about 140° C., or about 110° C. to about 120° C., or about 30° C. to about 110° C., or about 50° C. to about 90° C., or about 120° C. to about 200° C., or about 140° C. to about 180° C. In some aspects, the temperature is higher than the glass transition temperature of the polymer. In some aspects, the temperature is higher than the melting point of the polymer.

In some aspects, the method of recycling a polymer may be performed by the use of microwave irradiation. Microwaves act as high frequency electric fields and will generally heat any material containing mobile electric charges. Component molecules are forced to rotate with the field and lose energy in collisions. Acting as an internal heat source, microwave absorption is able to heat the target compounds without heating the entire furnace or oil bath, which saves time and energy. Numerous microwave reactors in which the above processes can be performed are commercially available and would be familiar to a person of ordinary skill in the art.

In some aspects, the method of recycling a polymer is performed in a batch reactor, a stirred-tank reactor, or an extrusion reactor. In some aspects, the method of recycling a polymer produces a compound of Formula I.

In some aspects, steps a) and b) do not occur concurrently. In some aspects, steps a) and b) do occur concurrently.

In some aspects, the method of recycling a polymer requires less energy inputs relative to other methods of recycling polymers. In some aspects, the method of recycling a polymer generates reduced waste streams during the process. In some aspects, the method of recycling a polymer generates a product with broader utility than other methods of recycling polymers. In some aspects, the method of recycling a polymer has more ambient operating conditions than other methods of recycling polymers. In some aspects, the method of recycling a polymer is more suitable for polymer streams that include physical contaminants (e.g., dirt, dyes, glues, etc.) than other methods of recycling polymers.

In another aspect, provided is a resin produced from any of the disclosed methods of recycling a polymer.

In another aspect, provided is a method of producing a plastic comprising crosslinking any of the disclosed resins. In some aspects, the step of crosslinking comprises heating the resin. In some aspects, the step of crosslinking comprises exposing the resin to ultraviolet light. In some aspects, the step of crosslinking comprises modifying pH of the resin.

In some aspects, the method of producing a plastic further comprises additive manufacturing or injection molding using any of the disclosed resins.

### EXEMPLARY ASPECTS

In view of the described processes and compositions, hereinbelow are described certain more particularly described aspects of the disclosures. These particularly recited aspects should not, however, be interpreted to have any limiting effect on any different claims containing different or more general teachings described herein or that the “particular” aspects are somehow limited in some way other than the inherent meanings of the composites and formulas literally used therein.

Example 1: A Compound of Formula I:

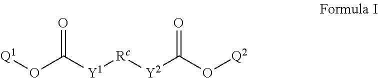

wherein: R^{c} is C₁₋₁₆ aliphatic group; each of Y¹ and Y² is independently selected at each occurrence from null, NH, O, and S; each of Q¹ and Q² is independently selected at each occurrence from Formula IIa and Formula IIb:

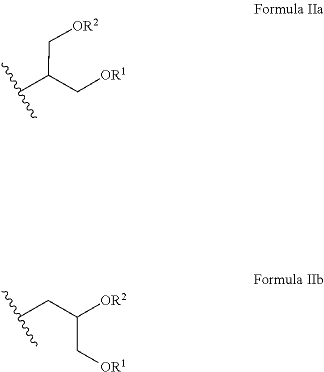

wherein R¹ is selected from R^{1a} or C(O)R^{1a}, R² is selected from R^{2a} or C(O)R^{1a}, R^{1a} is selected from H, C₁₋₁₂ alkyl, C₂₋₁₂ alkenyl, C₂₋₁₂ alkynyl, aryl, heterocyclyl, or heteroaryl; wherein R^{1a} may be substituted one or more times by F, Cl, Br, I, OH, SH, C(═O)OₓR^{1b}, aryl, heteroaryl, N₃, or N(R^{1b})₂, wherein x is 0 or 1, R^{1b} is independently selected from H, C₁₋₄ alkyl, and C₂₋₄ alkenyl; R^{2a} is selected from H, C₁₋₁₂ alkyl, C₂₋₁₂ alkenyl, C₂₋₁₂ alkynyl, aryl, heterocyclyl, or heteroaryl; wherein R^{2a} may be substituted one or more times by F, Cl, Br, I, OH, SH, C(═O)O_{y}R^{2b}, aryl, heteroaryl, N₃, or N(R^{2b})₂, wherein y is 0 or 1, R^{2b} is independently selected from H, C₁₋₄ alkyl, and C₂₋₄ alkenyl; wherein, for at least one of Q¹ and Q², at least one of R¹ and R² can participate in a polymerization reaction.

Example 2: The compound of any examples herein, wherein R¹ is a group that can participate in a polymerization reaction and R¹ has the formula:

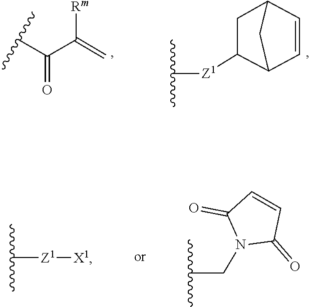

wherein R^{m} is selected from H and CH₃; Z¹ is selected from null, C₁₋₈ n-alkylene or C₁₋₈ heteroalkylene; X¹ is selected from CH═CH₂, C≡CH, N₃, SH; and R² is H or C₁₋₄ alkyl, optionally substituted one or more times by F, Cl, C₁₋₄ alkoxy, or aryl.

Example 3: The compound of any examples herein, wherein R² is a group that can participate in a polymerization reaction and R² has the formula:

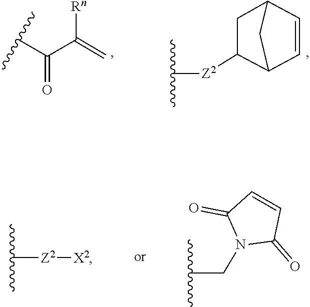

wherein Rⁿ is selected from H and CH₃; Z² is selected from null, C₁₋₈ alkylene or C₁₋₈ heteroalkylene; X² is selected from CH═CH₂, C≡CH, N₃, SH; and R¹ is C₁₋₄ alkyl, optionally substituted one or more times by F, Cl, C₁₋₄ alkoxy, or aryl.

Example 4: The compound of any examples herein, wherein R¹ has the formula:

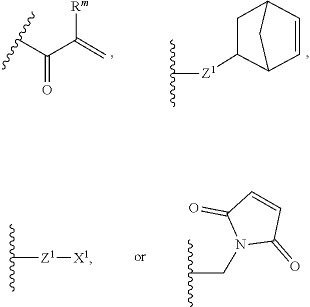

wherein R^{m} is selected from H and CH₃; Z¹ is selected from null, C₁₋₈ alkylene or C₁₋₈ heteroalkylene; X¹ is selected from CH═CH₂, C≡CH, N₃, SH; and R² has the formula:

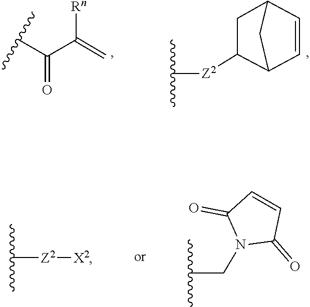

wherein R″ is selected from H and CH₃; Z² is selected from null, C₁₋₈ alkylene or C₁₋₈ heteroalkylene; X² is selected from CH═CH₂, C≡CH, N₃, SH.

Example 5: The compound of any examples herein, wherein R¹ is:

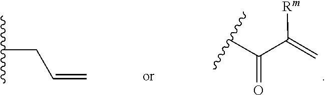

Example 6: The compound of any examples herein, wherein R² is:

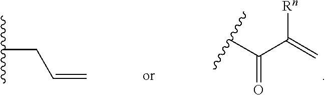

Example 7: The compound of any examples herein, wherein Y¹ and Y² are both null, or Y¹ and Y² are both NH.

Example 8: The compound of any examples herein, wherein Re is C₂₋₈ n-alkyl, C₂₋₈ alkenyl, or Re has the formula:

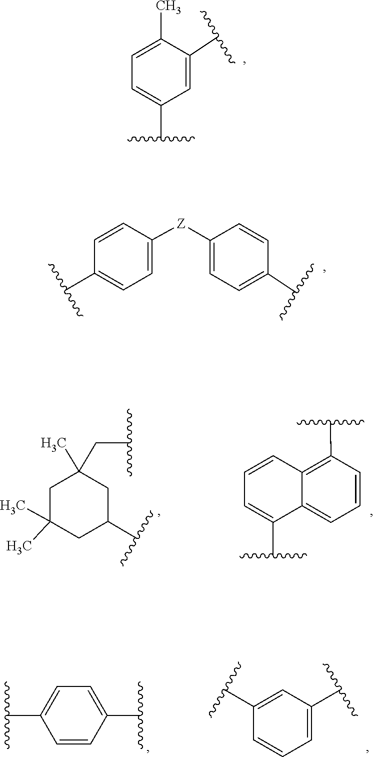

wherein Z is selected from null, CH₂, C(CH₃)₂, O, NH.

Example 9: The compound of any examples herein, wherein R^{c} is

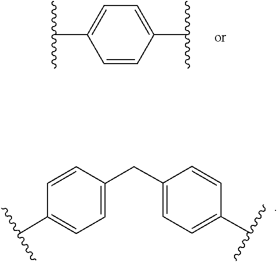 [SMILES: *c1ccc(*)cc1 ; *c1ccc(Cc2ccc(*)cc2)cc1]

Example 10: A method of recycling a polymer into a resin/prepolymer comprising contacting a polymer with a compound of Formula IIIa or Formula IIIb:

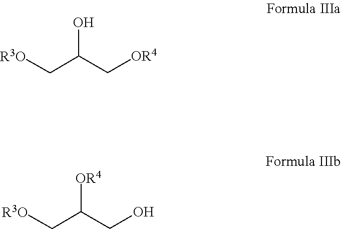

wherein R³ is selected from R^{3a} or C(O)R^{3a}, R⁴ is selected from R^{4a} or C(O)R^{4a}, R^{3a} is selected from H, C₁₋₁₂ alkyl, C₂₋₁₂ alkenyl, C₂₋₁₂ alkynyl, aryl, heterocyclyl, or heteroaryl; wherein R^{3a} may be substituted one or more times by F, Cl, Br, I, OH, SH, C(═O)OₓR^{3b}, aryl, heteroaryl, N₃, or N(R^{3b})₂, wherein x is 0 or 1, R^{3b} is independently selected from H, C₁₋₄ alkyl, and C₂₋₄ alkenyl; R^{4a} is selected from H, C₁₋₁₂ alkyl, C₂₋₁₂ alkenyl, C₂₋₁₂ alkynyl, aryl, heterocyclyl, or heteroaryl; wherein R^{4a} may be substituted one or more times by F, Cl, Br, I, OH, SH, C(═O)O_{y}R^{4b}, aryl, heteroaryl, N₃, or N(R^{4b})₂, wherein y is 0 or 1, R^{4b} is independently selected from H, C₁₋₄ alkyl, and C₂₋₄ alkenyl; and wherein at least one of R³ or R⁴ can participate in a polymerization reaction.

Example 11: The method of recycling a polymer of any examples herein, wherein R³ has the formula:

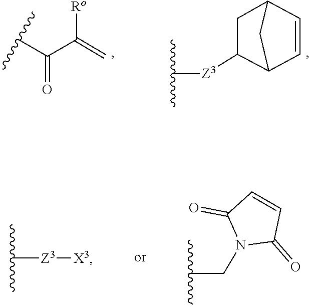

wherein R^{o} is selected from H and CH₃; Z³ is selected from null, C₁₋₈ n-alkylene; X³ is selected from CH═CH₂, C≡CH, N₃, SH; and R⁴ is C₁₋₄ alkyl, optionally substituted one or more times by F, Cl, C₁₋₄ alkoxy, or aryl.

Example 12: The method of recycling a polymer of any examples herein, wherein R⁴ has the formula:

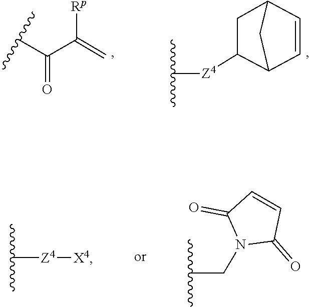

wherein R^{p} is selected from H and CH₃; Z⁴ is selected from null, C₁₋₈ alkylene or C₁₋₈ heteroalkylene; X⁴ is selected from CH═CH₂, C≡CH, N₃, SH; and R³ is H or C₁₋₄ alkyl, optionally substituted one or more times by F, Cl, C₁₋₄ alkoxy, or aryl.

Example 13: The method of recycling a polymer of any examples herein, wherein R³ has the formula:

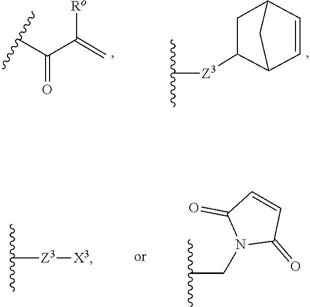

wherein R° is selected from H and CH₃; Z³ is selected from null, C₁₋₈ alkylene or C₁₋₈ heteroalkylene; X³ is selected from CH—CH₂, C≡CH, N₃, SH; and R⁴ has the formula:

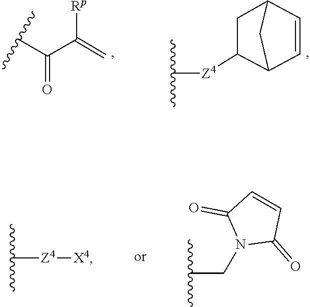

wherein R^{p} is selected from H and CH₃; Z⁴ is selected from null, C₁₋₈ alkylene or C₁₋₈ heteroalkylene; X⁴ is selected from CH═CH₂, C≡CH, N₃, SH.

Example 14: The method of recycling a polymer of any examples herein, wherein R³ is

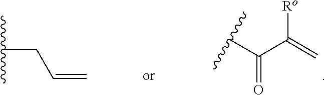

Example 15: The method of recycling a polymer of any examples herein, wherein R⁴ is

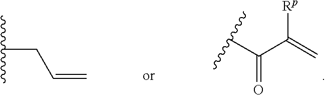

Example 16: The method of recycling a polymer of any examples herein, wherein the polymer comprises polyethylene terephthalate, polyester, polyurethane, polyesteramide, polyamide, epoxy resin, or any combination thereof.

Example 17: The method of recycling a polymer of any examples herein, wherein the compound of Formula III cleaves a bond between monomeric subunits of the polymer.

Example 18: The method of recycling a polymer of any examples herein, wherein the method produces a compound of Formula I.

Example 19: A method of recycling a polymer into a resin/prepolymer comprising a) contacting a polymer with a compound of Formula IV:

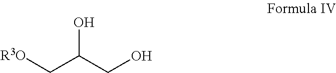

and b) contacting a product of the polymer and the compound of Formula IV with a compound of Formula V:

L—R⁴ Formula V

wherein R³ is selected from R^{3a} or C(O)R^{3a}, R⁴ is selected from R^{4a} or C(O)R⁴ª, L is a leaving group, R^{3a} is selected from H, C₁₋₁₂ alkyl, C₂₋₁₂ alkenyl, C₂₋₁₂ alkynyl, aryl, heterocyclyl, or heteroaryl; wherein R^{3a} may be substituted one or more times by F, Cl, Br, I, OH, SH, C(═O)OₓR^{3b}, aryl, heteroaryl, N₃, or N(R^{3b})₂, wherein x is 0 or 1, R^{3b} is independently selected from H, C₁₋₄ alkyl, and C₂₋₄ alkenyl; R^{4a} is selected from C₁₋₁₂ alkyl, C₂₋₁₂ alkenyl, C₂₋₁₂ alkynyl, aryl, heterocyclyl, or heteroaryl; wherein R^{4a} may be substituted one or more times by F, Cl, Br, I, OH, SH, C(═O)O_{y}R^{4b}, aryl, heteroaryl, N₃, or N(R^{4b})₂, wherein y is 0 or 1, R^{4b} is independently selected from H, C₁₋₄ alkyl, and C₂₋₄ alkenyl; and wherein R⁴ can participate in a polymerization reaction.

Example 20: The method of recycling a polymer of any examples herein, wherein the compound of Formula V is selected from:

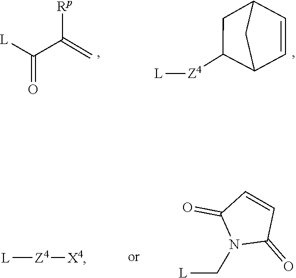

wherein L is a leaving group; R^{p} is selected from H and CH₃; Z⁴ is selected from null, C₁₋₈ alkylene or C₁₋₈ heteroalkylene; X⁴ is selected from CH═CH₂, C≡CH, N₃, SH; and R³ is C₁₋₄ alkyl, optionally substituted one or more times by F, Cl, C₁₋₄ alkoxy, or aryl.

Example 21: The method of recycling a polymer of any examples herein, wherein the compound of Formula V is

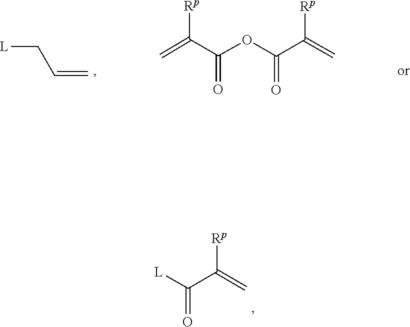

and L is OH, Cl, Br, I, OC₆F₅, OSO₃R^{s}, wherein R^{s} is methyl, trifluoromethyl, or 4-methylphenyl.

Example 22: The method of recycling a polymer of any examples herein, wherein the polymer comprises polyethylene terephthalate, polyester, polyurethane, polyesteramide, polyamide, epoxy resin, or any combination thereof.

Example 23: The method of recycling a polymer of any examples herein, wherein the compound of Formula IV cleaves a bond between monomeric subunits of the polymer.

Example 24: The method of recycling a polymer of any examples herein, wherein a transesterification reaction occurs between one of the OH groups of the compound of Formula IV and the compound of Formula V.

Example 25: The method of recycling a polymer of any examples herein, wherein steps a) and b) do not occur concurrently.

Example 26: The method of recycling a polymer of any examples herein, wherein the method produces a compound of Formula I.

Example 27: A resin/prepolymer produced from the method of recycling a polymer of any examples herein.

Example 28: A method of producing a plastic comprising crosslinking the resin of any examples herein.

Example 29: The method of producing a plastic of any examples herein, wherein the step of crosslinking comprises heating the resin.

Example 30: The method of producing a plastic of any examples herein, wherein the step of crosslinking comprises exposing the resin to ultraviolet light.

Example 31: The method of producing a plastic of any examples herein, wherein the step of crosslinking comprises modifying pH of the resin.

Example 32: The method of producing a plastic of any examples herein, further comprising additive manufacturing or injection molding using the resin of any examples herein.

What is claimed is:

1. A compound of Formula I: 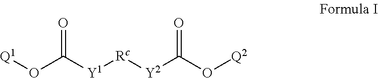
wherein:
R^{c} is C₁₋₁₆ aliphatic group;
each of Y¹ and Y² is independently selected at each occurrence from null, NH, O, and S;
each of Q¹ and Q² is independently selected at each occurrence from Formula IIa and Formula IIb: 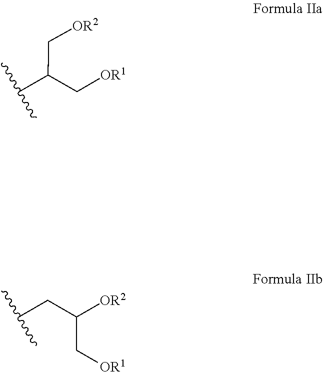
wherein
R¹ is selected from R^{1a} and C(O)R^{1a},
R² is selected from R^{2a} and C(O)R^{1a},
R^{1a} is selected from H, C₁₋₁₂ alkyl, C₂₋₁₂ alkenyl, C₂₋₁₂ alkynyl, aryl, heterocyclyl, and heteroaryl; wherein R^{1a} may be substituted one or more times by F, Cl, Br, I, OH, SH, C(═O)OₓR^{1b}, aryl, heteroaryl, N₃, or N(R^{1b})₂, wherein x is 0 or 1, R^{1b} is independently selected from H, C₁₋₄ alkyl, and C₂₋₄ alkenyl;
R^{2a} is selected from H, C₁₋₁₂ alkyl, C₂₋₁₂ alkenyl, C₂₋₁₂ alkynyl, aryl, heterocyclyl, and heteroaryl; wherein R^{2a} may be substituted one or more times by F, Cl, Br, I, OH, SH, C(═O)O_{y}R^{2b}, aryl, heteroaryl, N₃, or N(R^{2b})₂, wherein y is 0 or 1, R^{2b} is independently selected from H, C₁₋₄ alkyl, and C₂₋₄ alkenyl;
wherein, for at least one of Q¹ and Q², at least one of R¹ and R² can participate in a polymerization reaction.

2. The compound of claim 1, wherein R¹ has the formula: 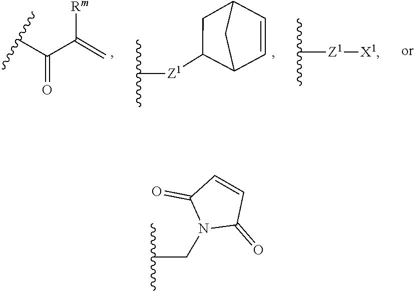
wherein
R^{m} is selected from H and CH₃;
Z¹ is selected from null, C₁₋₈ n-alkylene, and C₁₋₈ heteroalkylene;
X¹ is selected from CH═CH₂, C≡CH, N₃, and SH; and
R² is H or C₁₋₄ alkyl, optionally substituted one or more times by F, Cl, C₁₋₄ alkoxy, or aryl.

3. The compound of claim 1, wherein R² has the formula: 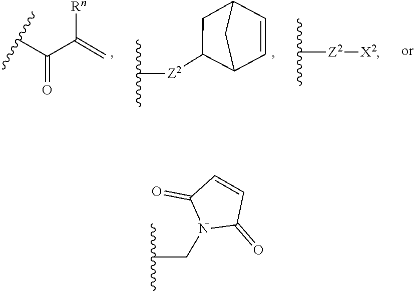
wherein
Rⁿ is selected from H and CH₃;
Z² is selected from null, C₁₋₈ alkylene, and C₁₋₈ heteroalkylene;
X² is selected from CH═CH₂, C≡CH, N₃, and SH; and
R¹ is C₁₋₄ alkyl, optionally substituted one or more times by F, Cl, C₁₋₄ alkoxy, or aryl.

4. The compound of claim 1, wherein R¹ has the formula: 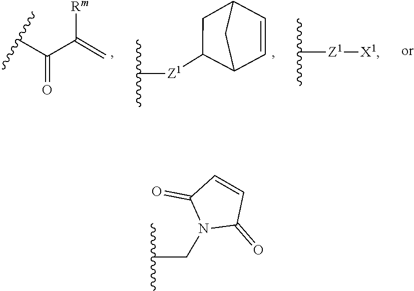
wherein R^{m} is selected from H and CH₃;
Z¹ is selected from null, C₁₋₈ alkylene, and C₁₋₈ heteroalkylene;
X¹ is selected from CH═CH₂, C≡CH, N₃, and SH; and
R² has the formula: 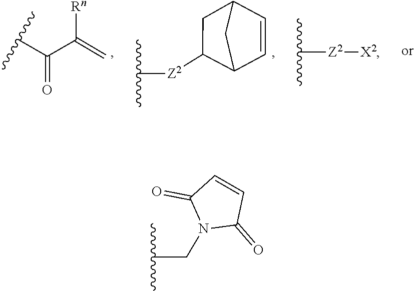
wherein
Rⁿ is selected from H and CH₃;
Z² is selected from null, C₁₋₈ alkylene, and C₁₋₈ heteroalkylene;
X² is selected from CH═CH₂, C≡CH, N₃, and SH.

5. The compound of claim 1, wherein R¹ is:

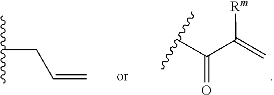

6. The compound of claim 1, wherein R² is:

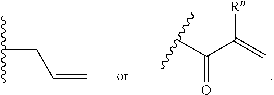

7. The compound of claim 1, wherein Y¹ and Y² are both null, or Y¹ and Y² are both NH.

8. The compound of claim 1, wherein R^{c} is C₂₋₈ n-alkyl, C₂₋₈ alkenyl, or Re has the formula: 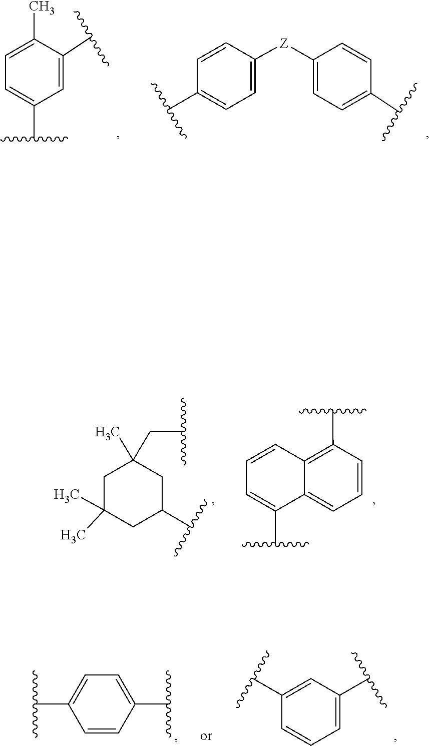
wherein Z is selected from null, CH₂, C(CH₃)₂, O, and NH.

9. A method of recycling a polymer into a resin/prepolymer comprising contacting a polymer with a compound of Formula IIIa, Formula IIIb, or a combination thereof: 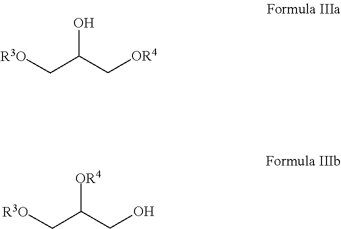
wherein
R³ is selected from R^{3a} and C(O)R^{3a},
R⁴ is selected from R^{4a} and C(O)R^{4a},
R^{3a} is selected from H, C₁₋₁₂ alkyl, C₂₋₁₂ alkenyl, C₂₋₁₂ alkynyl, aryl, heterocyclyl, and heteroaryl; wherein R^{3a} may be substituted one or more times by F, Cl, Br, I, OH, SH, C(═O)OₓR^{3b}, aryl, heteroaryl, N₃, or N(R^{3b})₂, wherein x is 0 or 1, R^{3b} is independently selected from H, C₁₋₄ alkyl, and C₂₋₄ alkenyl;
R^{4a} is selected from H, C₁₋₁₂ alkyl, C₂₋₁₂ alkenyl, C₂₋₁₂ alkynyl, aryl, heterocyclyl, and heteroaryl; wherein R^{4a} may be substituted one or more times by F, Cl, Br, I, OH, SH, C(═O)O_{y}R^{4b}, aryl, heteroaryl, N₃, or N(R^{4b})₂, wherein y is 0 or 1, R^{4b} is independently selected from H, C₁₋₄ alkyl, and C₂₋₄ alkenyl; and
wherein at least one of R³ or R⁴ can participate in a polymerization reaction.

10. The method of recycling a polymer of claim 9, wherein R³ has the formula: 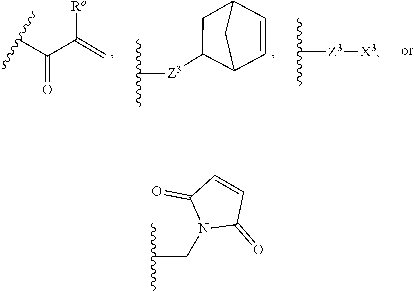
wherein
R^{o} is selected from H and CH₃;
Z³ is selected from null and C₁₋₈ n-alkylene;
X³ is selected from CH═CH₂, C≡CH, N₃, and SH; and
R⁴ is C₁₋₄ alkyl, optionally substituted one or more times by F, Cl, C₁₋₄ alkoxy, or aryl.

11. The method of recycling a polymer of claim 9, wherein R⁴ has the formula: 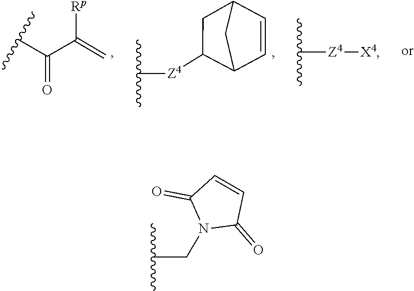
wherein
R^{p} is selected from H and CH₃;
Z⁴ is selected from null, C₁₋₈ alkylene, and C₁₋₈ heteroalkylene;
X⁴ is selected from CH═CH₂, C≡CH, N₃, and SH; and
R³ is H or C₁₋₄ alkyl, optionally substituted one or more times by F, Cl, C₁₋₄ alkoxy, or aryl.

12. The method of recycling a polymer of claim 9, wherein R³ has the formula: 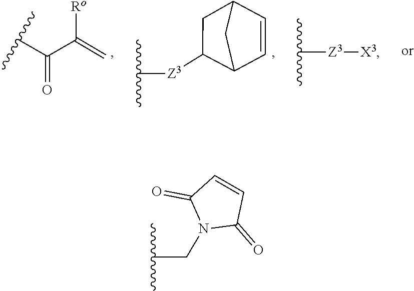
wherein
R^{o} is selected from H and CH₃;
Z³ is selected from null, C₁₋₈ alkylene, and C₁₋₈ heteroalkylene;
X³ is selected from CH═CH₂, C≡CH, N₃, and SH; and
R⁴ has the formula: 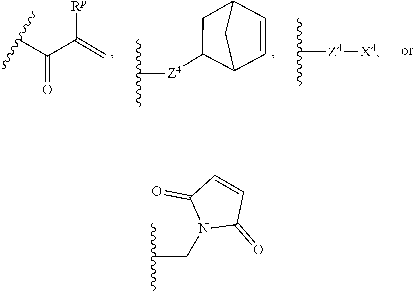
wherein
R^{P} is selected from H and CH₃;
Z⁴ is selected from null, C₁₋₈ alkylene, and C₁₋₈ heteroalkylene;
X⁴ is selected from CH═CH₂, C≡CH, N₃, and SH.

13. The method of recycling a polymer of claim 9, wherein the polymer comprises polyethylene terephthalate, polyester, polyurethane, polyesteramide, polyamide, epoxy resin, or any combination thereof.

14. The method of recycling a polymer of claim 9, wherein the method produces a compound of Formula I.

15. A method of recycling a polymer into a resin/prepolymer comprising a) contacting a polymer with a compound of Formula IV: 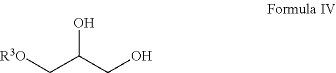
and b) contacting a product of the polymer and the compound of Formula IV with a compound of Formula V:
L—R⁴ Formula V
wherein
R³ is selected from R^{3a} and C(O)R^{3a},
R⁴ is selected from R^{4a} and C(O)R^{4a},
L is a leaving group,
R^{3a} is selected from H, C₁₋₁₂ alkyl, C₂₋₁₂ alkenyl, C₂₋₁₂ alkynyl, aryl, heterocyclyl, and heteroaryl; wherein R^{3a} may be substituted one or more times by F, Cl, Br, I, OH, SH, C(═O)OₓR^{3b}, aryl, heteroaryl, N₃, or N(R^{3b})₂, wherein x is 0 or 1, R^{3b} is independently selected from H, C₁₋₄ alkyl, and C₂₋₄ alkenyl;
R^{4a} is selected from C₁₋₁₂ alkyl, C₂₋₁₂ alkenyl, C₂₋₁₂ alkynyl, aryl, heterocyclyl, and heteroaryl; wherein R^{4a} may be substituted one or more times by F, Cl, Br, I, OH, SH, C(═O)O_{y}R^{4b}, aryl, heteroaryl, N₃, or N(R^{4b})₂, wherein y is 0 or 1, R^{4b} is independently selected from H, C₁₋₄ alkyl, and C₂₋₄ alkenyl; and
wherein R⁴ can participate in a polymerization reaction.

16. The method of recycling a polymer of claim 15, wherein the compound of Formula V is selected from: 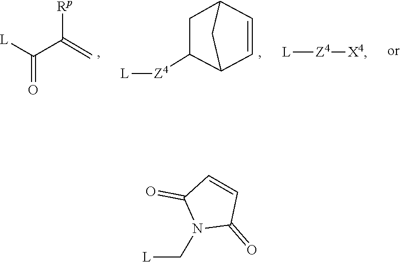
wherein
L is a leaving group;
R^{p} is selected from H and CH₃;
Z⁴ is selected from null, C₁₋₈ alkylene, and C₁₋₈ heteroalkylene;
X⁴ is selected from CH═CH₂, C≡CH, N₃, and SH; and
R³ is C₁₋₄ alkyl, optionally substituted one or more times by F, Cl, C₁₋₄ alkoxy, or aryl.

17. The method of recycling a polymer of claim 15, wherein the compound of Formula V is 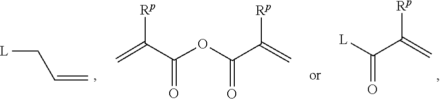
and L is OH, Cl, Br, I, OC₆F₅, or OSO₃R^{s}, wherein R^{s} is methyl, trifluoromethyl, or 4-methylphenyl.

18. The method of recycling a polymer of claim 15, wherein the polymer comprises polyethylene terephthalate, polyester, polyurethane, polyesteramide, polyamide, epoxy resin, or any combination thereof.

19. The method of recycling a polymer of claim 15, wherein steps a) and b) do not occur concurrently.

20. The method of recycling a polymer of claim 15, wherein the method produces a compound of Formula I.
